(19) United States
(12) Patent Application Publication
(10) Pub. No.: US 2005/0265958 A1
(43) Pub. Date: Dec. 1, 2005

(54) Nitric oxide releasing compositions and associated methods

(76) Inventors: Jennifer L. West, Pearland, TX (US); Lakeshia J. Taite, Grove Hill, AL (US)

Correspondence Address: Thomas M. Morrow;Baker Botts L.L.P., One Shell Plaza, 910 Louisiana Street, Houston, TX, 77002-4995, US

(21) Appl. No.: 11/129,883

(22) Filed: May 16, 2005

Related U.S. Application Data
(60) Provisional application No. 60/571,134, filed on May 14, 2004.

(51) Int. Cl. A61K 31/785

(52) U.S. Cl. 424/78.27

(57) ABSTRACT

Dendritic nitric oxide donors having the formula:
[P]-[(A)_{y}]ₓ-[(NO)_{z}]_{q} wherein P is a core that comprises a biocompatible polymer; A is a branching unit monomer that comprises at least one end group capable of reversibly attaching NO; (NO) is nitric oxide; x, y, and z are positive integers greater than or equal to 1; and q is a positive integer greater than or equal to y, as well as medical devices and kits that comprise dendritic nitirc oxide donors, are provided. Also provided are methods of delivering nitric oxide into a recipient subject comprising: providing a dendritic nitric oxide donor; and administering the dendritic nitric oxide donor into the recipient subject, such that the dendritic nitric oxide donor releases NO in the recipient subject.

### CROSS-REFERENCE TO RELATED APPLICATION

This Application claims priority to U.S. Provisional Patent Application Ser. No. 60/571,134 filed on May 14, 2004 and entitled “Nitric Oxide Releasing Compositions and Methods.”

### STATEMENT OF GOVERNMENT INTEREST

The present invention was developed under grants from the National Science Foundation (Grant Nos. HRD-9817555 and 0114254). The U.S. Government may have certain rights to the invention.

### BACKGROUND

The present invention generally relates to compositions capable of releasing nitric oxide and methods of using such compositions.

Nitrogen monoxide can exist as various redox species with distinctive properties and reactivities. These species include: NO⁺ (nitrosonium), NO. (nitric oxide), and NO⁻ (nitroxyl anion). Of these species, nitric oxide (commonly referred to as NO) has been implicated in a wide range of biological functions. As a result, NO, and materials that release NO, are candidate therapeutics for a range of diverse disease states.

For example, NO is associated with the maintenance of vascular homeostasis (e.g., vascular endothelial cells may produce NO that regulates vasomotor tone, inhibits vascular smooth muscle cell proliferation, and inhibits platelet adhesion to the vasculature). In addition, studies have shown that endothelial cells in the inner intimal layer of an artery produce NO in response to shear stress or other vasodilatory stimuli. Additionally, endothelial NO may cause vascular smooth muscle to relax and allow dilation to occur. NO also may inhibit platelet aggregation and adhesion.

NO also has been shown to play a role in restenosis, which is described as the reocclusion of blood vessels after the treatment of coronary artery stenosis by percutaneous transluminal coronary angioplasty (PTCA). Restenosis is typically characterized as a healing response that occurs over a period of months following the injury caused by PTCA. This healing response typically results in reduced arterial expansion and platelet adhesion and aggregation. Restenosis is a serious clinical concern in the treatment of coronary artery disease.

No fully established therapy exists to prevent restenosis, but a number of treatments are widely used. For example, coronary stenting has seen considerable use since its introduction in 1986. Though potentially useful in preventing elastic recoil of the artery, the implantation of coronary stents may be problematic. Wall injury at the site of stent deployment can lead to platelet activation and thrombus formation. Furthermore, the surface of the stent also is thrombogenic, leading to an increased risk of thrombosis soon after treatment by PTCA. Thus, potential for in-stent restenosis exists.

Endoluminal paving (e.g., coating the interior of the arterial surface with a solid paving of polymer gel) also has shown potential in deterring luminal narrowing after injury. Thin hydrogel barriers polymerized intravascularly after stent deployment have been shown to reduce neointimal formation and thrombogenicity at the arterial wall. Hydrogel barriers used alone also have shown efficacy in eliminating thrombosis when tested on rats with a carotid artery crush injury, and in inhibiting thrombosis and intimal thickening when tested on rabbits with a balloon injury.

The use of systemic agents (e.g., antithrombotics, antiproliferative and antimigration drugs, and vasodilators) to treat restenosis has been evaluated in animal models, but no meaningful decrease in the incidence of restenosis was shown. Furthermore, systemic treatments in humans may be problematic due to the toxicity of certain agents.

An alternative to systemic treatment is local drug delivery. For example, catheters that release drugs either by diffusion- or pressure-driven mechanisms have been used to deliver doses of drugs to the treatment area before, or immediately after, denudation. Treatment via catheter administration, however, suffers from low efficiency and delocalization of the treatment material. Stents coated with drug-eluting polymers and stents made of biodegradable polymers loaded with drugs or genetic materials have been used as a means for local drug delivery. In some studies, such delivery means have suppressed neointimal formation.

Dendrimers may be used as a drug delivery system in a variety of applications. In general, dendrimers are synthetic, monodisperse macromolecules of nanometer dimensions, having a highly branched three-dimensional architecture in which bonds radiate from a central core. The main components of a dendrimer typically include a core, branching units, and end groups. Dendrimers typically are produced in an iterative sequence of reaction steps, in which each additional iteration leads to a higher generation dendrimer, with an increased number of end groups, and an increased molecular weight. Two general techniques are used to form dendrimers: divergent and convergent synthesis. In the divergent method, the dendrimer is assembled from the core to the periphery; in the convergent method, the dendrimer is synthesized beginning from the outside and terminating at the core.

Dendrimers often have several characteristics that make them attractive for biological and drug delivery applications. For example, dendrimers may have, among other things, a generally uniform size, water-solubility, internal cavities, and variable surface functionality. Dendrimers may be capable of possessing two major chemical environments. One major chemical environment may be supported on the surface of the dendrimer, and may be influenced by the surface chemistry among end groups. Another independent chemical environment may be found in the interior of the dendrimer, which may be shielded from exterior environments. Additionally, certain hydrophobic/hydrophilic and polar/nonpolar interactions may be varied in the two environments. The internal cavities present in some dendrimers also may be capable of containing guest molecules. The term “guest molecule” refers to molecules enclosed, in whole or in part, within the dendrimer.

Dendrimers have been used, among other things, as molecular weight and size standards, as gene transfection agents, as hosts for the transport of biologically important guest molecules, and as anti-cancer agents. Studies suggest that the structure of dendrimers may allow for improved drug loading and controlled release. For example, dendrimers have been studied in conjunction with the nonsteroidal antiinflammatory drug indomethacin, and with the anticancer drugs methotrexate, adriamycin, and taxol. Antibody-dendrimer conjugates also have been shown to retain immunoreactivity and to display high-binding specificity. The ability to functionalize surface groups and encapsulate guest molecules makes dendrimers suitable systems for drug delivery and offers the opportunity for targeted therapeutics.

Known methods of NO delivery include soluble, short-term NO donors, such as S-nitroso-N-acetyl-D,L-penicillamine (SNAP) and incorporation of NO donors into polymeric matrices. In general, NO-nucleophile complexes (e.g., diazeniumdiolate ions) and NO-donating groups (e.g., S-nitrosothiols) may spontaneously decompose in aqueous environments, such as physiological or bodily fluids, to release NO. This rapid, spontaneous decomposition, however, may not be a favorable property for many therapeutic applications. Generally, a slower rate of decomposition and more steady evolution of NO are more efficacious.

### SUMMARY

The present invention generally relates to compositions capable of releasing nitric oxide and methods of using such compositions.

According to one embodiment, the present invention provides a dendritic nitric oxide donor having the formula:
[P]-[(A)_{y}]ₓ-[(NO)_{z}]_{q}
wherein P is a core that comprises a biocompatible polymer; A is a branching unit monomer that comprises at least one end group capable of reversibly attaching NO; (NO) is nitric oxide; x, y, and z are positive integers greater than or equal to 1; and q is a positive integer greater than or equal to y.

According to another embodiment, the present invention provides a kit comprising at least one dendritic nitric oxide donor, wherein the dendritic nitric oxide donor comprises a compound having the formula:
[P]-[(A)_{y}]ₓ-[(NO)_{z}]_{q}
wherein P is a core that comprises a biocompatible polymer; A is a branching unit monomer that comprises at least one end group capable of reversibly attaching NO; (NO) is nitric oxide; x, y, and z are positive integers greater than or equal to 1; and q is a positive integer greater than or equal to y.

According to another embodiment, the present invention provides a medical device comprising at least one dendritic nitric oxide donor, wherein the dendritic nitric oxide donor comprises a compound having the formula:
[P]-[(A)_{y}]ₓ-[(NO)_{z}]_{q}
wherein P is a core that comprises a biocompatible polymer; A is a branching unit monomer that comprises at least one end group capable of reversibly attaching NO; (NO) is nitric oxide; x, y, and z are positive integers greater than or equal to 1; and q is a positive integer greater than or equal to y.

According to another embodiment, the present invention provides a method of delivering nitric oxide into a recipient subject comprising: administering a dendritic nitric oxide donor to a recipient subject, such that the dendritic nitric oxide donor releases NO in the recipient subject, the dendritic nitric oxide donor having the formula:
[P]-[(A)_{y}]ₓ-[(NO)_{z}]_{q}
wherein P is a core that comprises a biocompatible polymer; A is a branching unit monomer that comprises at least one end group capable of reversibly attaching NO; (NO) is nitric oxide; x, y, and z are positive integers greater than or equal to 1; and q is a positive integer greater than or equal to y.

The features and advantages of the present invention will be readily apparent to those skilled in the art upon a reading of the description of the embodiments that follows.

### BRIEF DESCRIPTION OF THE FIGURES

A more complete understanding of this disclosure may be acquired by referring to the following description taken in combination with the accompanying figures.

FIG. 1 is a graph illustrating NO release from a dendritic nitric oxide donor according to a specific example of the present invention.

FIG. 2 is a graph comparing endothelial cell proliferation and smooth muscle cell growth in the presence or absence (control) of a dendritic nitric oxide donor according to a specific example of the present invention.

FIG. 3 is a graph comparing the number of adherent platelets in the presence or absence (control) of a dendritic nitric oxide donor according to a specific example of the present invention.

FIG. 4 are digital photos of platelets fluorescently labeled with mepacrine showing the inhibition of platelet adhesion to thrombogenic surfaces by a dendritic nitric oxide donor according to a specific example of the present invention taken with a Nikon CoolPix 5000 camera (Nikon Corporation, Tokyo, Japan) under 200× magnification using a Zeiss Axiovert 135 microscope (Carl Zeiss Microimaging, Inc., Thomwood, N.Y.).

FIG. 5 is a graph illustrating NO release from a diazeniumdiolate ion comprising three lysines.

FIG. 6 is a graph illustrating NO release from a diazeniumdiolate ion comprising five lysines.

FIG. 7 is a graph illustrating NO release from a S-nitrosothiol comprising cysteine.

FIG. 8 is a diagram illustrating a scheme for targeting a dendritic nitric oxide donor according to a specific example of the present invention.

FIG. 9 are digital photos of a fluorescein 5-isothiocyanate (FITC)-labeled a dendritic nitric oxide donor according to a specific example of the present invention bound to human umbilical vein endothelial cells (HUVECs) taken with a Nikon CoolPix 5000 camera (Nikon Corporation, Tokyo, Japan) under 200× magnification using a Zeiss Axiovert 135 microscope (Carl Zeiss Microimaging, Inc., Thomwood, N.Y.) in which: A) shows FITC-labeled sialyl-Lewis-X conjugated dendritic nitric oxide donors with IL-1β stimulated HUVECs; B) shows sialyl-Lewis-X conjugated dendritic nitric oxide donors with unstimulated HUVECs; and C) shows FITC-labeled dendritic nitric oxide donors that have not been conjugated to sialyl-Lewis-X with IL-1β stimulated HUVECs.

The patent or application file contains at least one drawing executed in color. Copies of this patent or patent application publication with color drawing(s) will be provided by the Patent Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as defined by the appended claims.

### Description

The present invention generally relates to compositions capable of releasing nitric oxide and methods of using such compositions.

Dendritic Nitric Oxide Donor Compositions of the Present Invention.

The present invention provides dendritic nitric oxide donors that are capable of releasing nitric oxide under physiological conditions. The term “physiological conditions” refers to the conditions (e.g., pH and temperature) that may exist in a recipient subject. The dendritic nitric oxide donors of the present invention generally comprise a core to which multiple branching units may be attached; one or more branching units that are directly attached to and that extend from the core; and an end group derivatized with nitric oxide. As used herein, “attach,” “attachment,” “bind,” and “bound” may include, but are not limited to, such attachments as a covalent bond or an ionic bond. Certain embodiments of the dendritic nitric oxide donors of the present invention have been tailored from starting materials that are innately biocompatible, such as amino acids, proteins, and polysaccharides, and that generally form nontoxic nitrosation products. The term “biocompatible” refers to the property of being biologically compatible by not producing a significant toxic, injurious, or immunological response in living tissue.

In certain embodiments, the present invention provides dendritic nitric oxide donors represented by Formula (I):
[P]-[(A)_{y}]ₓ-[(NO)_{z}]_{q}
wherein P is a core that comprises a biocompatible polymer; A is a branching unit monomer that comprises at least one end group capable of reversibly attaching NO; (NO) is nitric oxide; x, y and z are positive integers greater than or equal to 1; and q is a positive integer greater than or equal to y.

In certain embodiments, P in Formula (I) may comprise one or more low-molecular-weight molecules with one or more functionalities capable of coupling (e.g., covalent and hydrogen bonding) to the focal point of a branching unit. In some embodiments, P may comprise a biocompatible polymer with one or more functional groups. The functional group may provide a means for attaching the branching units (e.g., [(A)_{y}]) to the core. In certain embodiments, P may comprise NO. For example, P may be capable of binding NO when P comprises a group that is capable of binding NO and the group is accessible for NO binding. In certain embodiments, P may further comprise a metal.

Examples of suitable biocompatible polymers include, but are not limited to, polyethylene glycol (PEG), poly(ethylenamine), poly(amidoamine) (PAMAM), polypropylenimine tetraamine, and the like. Examples of suitable biocompatible polymers may be found in Grayson and Frechet, Chemistry Reviews, Vol. 101 p. 3819 (2001). In certain embodiments in which a suitable biocompatible polymer does not already have a functional group, one may be chemically added. Such a chemical addition may be accomplished, with the benefit of this disclosure, using methods known in the art of preparative organic chemistry (e.g., reductive amination, preparation of amines through nucleophilic substitutions, and hydration of alkenes to hydroxyls). Suitable functional groups include, but are not limited to, amine groups, hydroxyl groups, N-hydroxysuccinimide esters, and carboxyl groups. In some embodiments, P in Formula (I) may include functionalized PEG molecules, such as methoxypoly(ethylene glycol)-amine, diaminopoly(ethylene glycol), PEG-N-hydroxysuccinimide ester monoacrylate, multi-arm PEGs, and MPEG-NHS. A variety of suitable functionalized PEG molecules are commercially available, including those supplied by Nektar Therapeutics, San Carlos, Calif.

In general, A in Formula (I) may be any biocompatible compound capable of releasing NO, or capable of being modified to release NO. In certain embodiments, A in Formula (I) may be capable of undergoing polymerization. Polymerization of A may result in the branching unit represented by [(A)_{y}] in Formula (I). A means for polymerization may be provided by one or more functional groups present on A. The specific functional groups, and number of functional groups, may be tailored to achieve a desired effect. For example, the number of functional groups may be tailored to increase the number of branching points for a given branching unit, thereby increasing y in Formula (I). Functional groups suitable as a means for polymerization include, but are not limited to, amine groups, carboxyl groups, thiol groups, hydroxyl groups, and the like.

In general, A in Formula (I) comprises at least one end group capable of reversibly attaching NO. In certain embodiments, NO may be released from the end group of A under physiological conditions of pH and temperature that are found in a recipient subject, e.g., a human. Suitable physiological conditions include pHs and temperatures that are within nonlethal limits for humans (see generally Guyton and Hall, Textbook of Medical Physiology, 10th Ed. (2000)). Suitable physiological pHs may be in the range of from about 6.8 to about 8.0. In certain embodiments, the physiological pH is in the range of from about 7.3 to about 7.5. Suitable physiological temperatures may be in the range of from about 65° F. to about 110° F. In certain embodiments, suitable physiological temperatures may be in the range of from about 98° F. to about 98.8° F. A nonlimiting theory to partially explain the release of NO from the dendritic nitric oxide donors of the present invention under physiological conditions is that dissociation of the NO is acid catalyzed and temperature dependant (see Keefer et al., Methods in Enzymology, Vol. 268 (1996)).

In general, when NO is released from the end group of A in Formula (I) the resultant compound should revert to a biocompatible molecule. In certain some embodiments, the end group of A in Formula (I) may be capable of forming NO-nucleophile complexes, such as, for example, diazeniumdiolate ions. In other embodiments, the end group of A in Formula (I) may be capable of forming NO-donating groups, such as, for example, S-nitrosothiols. Examples of such end groups include, but are not limited to, primary amines, thiols, ferrous nitro complexes, organic nitrites, and nitrates.

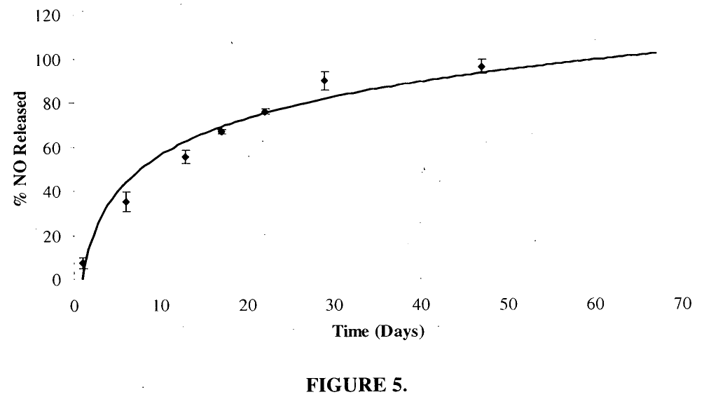

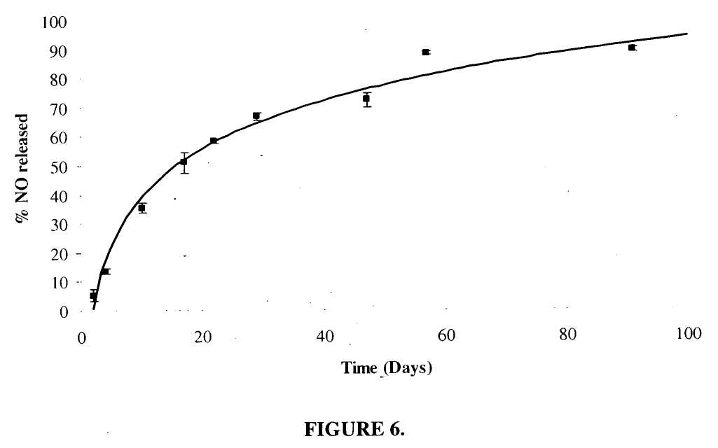

The choice of A provides a means for tailoring the nitric oxide release properties of the compositions of the present invention represented by Formula (I). For example, when A forms diazeniumdiolate ions, nitric acid may be released from the dendritic nitric oxide donor molecules of Formula (I) at a slower rate than when A forms S-nitrosothiols (compare FIGS. 5 and 6 to FIG. 7). Further, when A may form a diazeniumdiolate (e.g., when A is a lysine) and y is equal to one, NO may release on the order of minutes; but, when y is larger the release rate may be slower (compare FIG. 5 to FIG. 6). A nonlimiting theory to partially explain the difference in release rates may be that release of NO may depend, at least in part, on the molecular weight of the species (e.g., the larger the molecule, or the longer the chain, the slower the release) (see generally Hrabie et al., Journal of Organic Chemistry, 58:1472 (1996)).

Similarly, the specific choice of A also may affect the amount of NO that the dendritic nitric oxide donors of Formula (I) release. For example, each diazeniumdiolate ion that may be formed by an end group of A may be capable of releasing two NO molecules; whereas each S-nitrosothiol that may be formed by an end group of A may be capable of releasing only one NO molecule. In certain embodiments, the end group of A in Formula (I) may comprise other suitable NO donor complexes or NO-nucleophile complexes, for example, organic nitrites and nitrates, ferrous nitro complexes, or sydnonimines. The mechanisms of NO release may vary (e.g., enzymatic, chemical hydrolysis and/or chemical reduction) depending on the choice of A and the end group (see generally J. A. Bauer, et al., Advances in Pharmacology, 14:361 (1996)).

In certain embodiments, A comprises an amino acid. The amino acid may be a natural amino acid (e.g., glycine, alanine, valine, leucine, isoleucine, methionine, proline, phenylalanine, tryptophan, asparagine, glutamine, serine, threonine, aspartic acid, glutamic acid, tyrosine, cysteine, lysine, arginine, histidine, or combinations thereof). In certain embodiments in which A is lysine, its diamino nature provides a means to double the number of branching points with each generation. In certain embodiments where the branching unit is a heteropolymer, A may comprise independently both lysine and cysteine. In one example of such an embodiment, lysine may be used as the primary branching unit and cysteine added as the terminal group, leaving the thiol end groups of cysteine available to become S-nitrosothiols upon exposure to NO. In other embodiments, A may be diethyltriamine. The branching unit, [(A)_{y}] in Formula (I), may comprise the same branching unit monomer (e.g., lysine) or a combination of one or more different branching unit monomers (e.g., lysine and cysteine).

In certain embodiments, x in Formula (I) may be chosen based on the number of branching units desired, based on certain desired properties of the resultant compound, or both. In other embodiments, x may be chosen based on the number of functional groups present on a biocompatible polymer of P in Formula (I). In one embodiment, x is a positive integer in the range of from 1 to 12.

In certain embodiments, z in Formula (I) may be 1 or 2, depending on whether a S-nitrosothiol or a diazeniumdiolate is formed. For example, when z is 1 the composition represented by Formula (I) may be a S-nitrosothiol. And, when z is 2 the composition represented by Formula (I) may be a diazeniumdiolate ion.

In certain embodiments, y in Formula (I) may be chosen to achieve certain properties of the resultant compound. For example, when y is increased, the branching unit molecule becomes more complex. Branching units that are more complex and have a higher molecular weight generally have more end groups, thereby increasing the amount of NO payload of the resultant molecule. In general, mixtures of branching units of different lengths may be used. In one embodiment, y is a positive integer in the range of from about 2 to about 10.

In certain embodiments, q in Formula (I) depends on the number of branching unit monomers present. For example, when A is capable of binding one molecule of NO, q may equal y; and when A is capable of binding more than one molecule of NO, q may be greater than y.

In certain embodiments of the dendritic nitric oxide donors of the present invention represented by Formula (I), a practical upper limit may exist for x, y, z, and q. Such upper limit may be defined by the practicality of combining or adding molecules based on, for example, the properties of the resultant compound and the cost of producing the compound. With the benefit of this disclosure, the practical upper limit of x, y, z, q will be apparent to a person having ordinary skill in the art.

To determine the level of controlled nitric oxide release, the dendritic nitric acid donors of the present invention may be tested using in vitro assays, for example, designed to measure one or more of cell proliferation, cell adhesion, and NO release. Using such assays, dendritic nitric oxide donors having the desired biological activity may be readily identified.

In certain embodiments, the dendritic nitric oxide donors of the present invention may comprise a metabolically produced form of the compound represented by Formula (I). For example, after administration into a recipient subject (e.g., a human or an animal) a certain embodiment of a dendritic nitric oxide donor may partially degrade, or release NO, or both, thereby altering the chemical composition of the dendritic nitric oxide donor of Formula (I).

In certain embodiments, the present invention provides dendritic nitric oxide donors to which at least one targeting agent is operatively attached. The term “targeting agent” refers to any compound, ligand, or chemical moiety that may be directed to an organelle, cell, tissue, or organ. Targeting agents may be operatively attached to one or more branching units or branching unit monomers of the dendritic nitric oxide donors of the present invention represented by Formula (I). The term “operatively attached” is used herein to refer to any physical or chemical attachment such as, but not limited to, covalent or ionic bonding, London dispersion forces, or van der Waals forces. It is contemplated that any targeting agent may be used in the compositions and methods of the present invention, either alone or in combination. In certain embodiments, a branching unit may comprise a targeting agent, nitric oxide, or both. Targeting agents may be bound to the dendritic nitric oxide donors of the present invention through a multivalency cluster effect by conjugating multiple target-homing ligands. In some embodiments, a targeting agent may be bound to an end group. In other embodiments, targeting agents may be incorporated into cavities that may be formed by the branching units.

Various agents for targeting molecules to specific cells, tissue, organs, and organisms are known to those of ordinary skill in the art and may be used in the methods and compositions of the present invention. In certain nonlimiting examples, a targeting agent may comprise a protein, such as a receptor protein (e.g., complementarity determinant, such as CD4, CD8, annexin V, or soluble fragments thereof); an antibody; an antibody fragment; a peptide; a cytokine; a growth factor hormone; a lymphokine; a nucleic acid that binds corresponding nucleic acids through base pair complementarity; or a combination thereof. In still other embodiments, the targeting agent may comprise one or more of a cellular receptor-targeting ligand; a fusogenic ligand; a nucleus-targeting ligand (see, e.g., U.S. Pat. No. 5,908,777); and an integrin receptor ligand (see, e.g., U.S. Pat. No. 6,083,741). Other small molecules, or molecules that bind to a cell surface molecule, (e.g., folic acid), also may be used.

In certain embodiments in which a practitioner desires the ability to target dendritic nitric oxide donors of the present invention to stimulated endothelium, the targeting agent may comprise a compound capable of targeting a selectin. Examples of selectins include, but are not limited to, leukocyte-homing receptors (LAM-1, L-selectin), endothelial leukocyte adhesion molecules (ELAM-1, E-selectin), and CD62 (P-selectin) on platelets and endothelial cells. A nonlimiting example of a selectin-specific targeting agent is sialyl-Lewis-X, which may be used to recognize E-selectin.

In certain embodiments, the dendritic nitric oxide donors of the present invention may be used to deliver guest molecules for therapeutic benefit. For example, the compounds represented by Formula (I) may encapsulate guest molecules that have analogous or synergist effects with NO. Suitable agents include, but are not limited to, 3-(5′-hydroxymethyl-2′furyl)-1-benzyl indazole (YC-1) (CAS No.: 170632-47-0). A nonlimiting list of examples of suitable guest molecules and properties of suitable guest molecules may be found in Grayson and Frechet, Chemistry Reviews, 101:3819 (2001).

In certain embodiments, a dendritic nitric oxide donor of the present invention may be targeted by tailoring its overall charge to complement the charge of an organelle, a cell, a tissue, or an organ. In other embodiments in which the dendritic nitric oxide donors of the present invention are polycationic, they may be targeted to the extracellular matrix. A nonlimiting explanation of this sort of targeting is that the positively charged dendritic nitric oxide donor is attracted to the negatively charged extracellular matrix (see generally Sakharov et al., Arteriosclerosis, Thrombosis, and Vascular Biology, 21(6):943-8 (2001)).

In general, the dendritic nitric oxide donors of the present invention may be synthesized in three basic steps: (1) synthesis of branching unit; (2) synthesis of copolymer; and (3) NO addition. The synthesis of the dendritic nitric oxide donors of the present invention may be accomplished, with the benefit of this disclosure, using methods known in the art of preparative organic chemistry. Such methods may be found, for example, in Sadler and Tam, Reviews in Molecular Biotechnology 90:195-229 (2002); Grayson and Frechet, Chemistry Reviews, 101:3819-67 (2001); Saavedra et al., Journal of Medicinal Chemistry 39:436-65 (1996); and Hrabie and Klose, Journal of Organic Chemistry, 58:1472-76 (1993).

In certain embodiments, the dendritic nitric oxide donors of the present invention may be synthesized using liquid-phase peptide synthesis. According to this method, a peptide chain is grown while attached to a soluble protecting group, for example, PEG (see generally The Peptides: Analysis Synthesis, Biology, Vol. 2: Special Methods in Peptide Synthesis Part A. 286-332 (Academic Press, 1980)).

In other embodiments, the dendritic nitric oxide donors of the present invention may be synthesized using solid-phase peptide synthesis. According to this method, a peptide chain is grown while attached to an insoluble resin, thereby making excess, reagents and byproducts easier to remove.

Methods of the Present Invention.

The present invention provides methods for inhibiting cellular functions such as cell proliferation, aggregation, and adhesion. The methods of the invention are based on the observations, as exemplified in the working examples, that the dendritic nitric oxide donors of the present invention are capable of inhibiting cell proliferation, as well as cell adhesion.

The present invention also provides methods of treating diseases or physiological conditions that are associated with, or affected by, NO mediated cell proliferation aggregation or adhesion. Human or animal systems that may be affected by NO, include, for example, vascular, dermal, neural, pulmonary, endocrine, gastrointestinal, and urogenital systems.

In certain embodiments, the dendritic nitric oxide donors of the present invention may be used as a treatment or therapy for a cardiovascular disease or disorder such as restenosis, coronary artery disease, atherosclerosis, atherogenesis, cerebrovascular disease, angina, ischemic disease, congestive heart failure, pulmonary edema associated with acute myocardial infarction, thrombosis, high or elevated blood pressure in hypertension, platelet aggregation, platelet adhesion, smooth muscle cell proliferation, a vascular or nonvascular complication associated with the use of a medical device, a wound associated with the use of a medical device, vascular or nonvascular wall damage, peripheral vascular disease, or neointimal hyperplasia following percutaneous transluminal coronary angiograph.

In certain embodiments, the dendritic nitric oxide donors of the present invention may be used as a treatment or therapy for a pathological condition resulting from abnormal cell proliferation (e.g., a cancer, a Karposi's sarcoma, a cholangiocarcinoma, a choriocarcinoma, a neoblastoma, a Wilm's tumor, Hodgkin's disease, a melanoma, multiple myelomas, a chronic lymphocytic leukemia, or an acute or chronic granulocytic lymphoma); a transplant rejection, an autoimmune, inflammatory, proliferative, hyperproliferative or vascular disease (e.g., rheumatoid arthritis, restenosis, lupus erythematosus, systemic lupus erythematosus, Hashimotos thyroiditis, myasthenia gravis, diabetes mellitus, uveitis, nephritic syndrome, multiple sclerosis, an inflammatory skin disease, an inflammatory lung disease, an inflammatory bowel disease, an inflammatory disease that affects or causes obstruction of a body passageway, an inflammation of the eye, nose or throat, a fungal infection, or a food-related allergy); or for reducing scar tissue or for inhibiting wound contraction in a subject in need thereof.

Pharmaceutical Compositions of the Present Invention.

Pharmaceutical compositions of the present invention include an effective amount of one or more dendritic nitric oxide donors of the present invention alone, or in combination with any other drugs, therapeutic agents, diagnostic agents, polymers, or additional agents, dissolved or dispersed in a pharmaceutically acceptable medium. The phrases “pharmaceutical” and “pharmaceutically acceptable” refer to molecular entities and compositions that do not tend to produce an adverse, allergic, or other untoward reaction when appropriately administered to human or an animal. The preparation of a pharmaceutical composition will be known to those of skill in the art in light of the present disclosure, as exemplified by Remington 's Pharmaceutical Sciences, 18th Ed. (Mack Printing Company 1990). Moreover, compositions of the present invention that are intended to be administered to a human or an animal should meet sterility, pyrogenicity, general safety, and purity standards as required by the FDA Office of Biological Standards. The dosage, formulation, and delivery may be selected for a particular therapeutic application (e.g., aerosols for respiratory tract delivery as described in I. Gonda, Critical Reviews in Therapeutic Drug Carrier Systems, 6:273-13 (1990)).

As used herein, “pharmaceutically acceptable medium” includes any and all carriers, solvents, dispersion media, coatings, surfactants, antioxidants, preservatives (e.g., antibacterial agents, antifungal agents), isotonic agents, absorption-delaying agents, salts, preservatives, drugs, drug stabilizers, binders, excipients, disintegration agents, lubricants, sweetening agents, flavoring agents, dyes, and such like materials, and combinations thereof (see, e.g., Remington's Pharmaceutical Sciences, supra at 1289-29). Except insofar as any conventional carrier is incompatible with the active ingredient, its use in the pharmaceutical compositions of the present invention is contemplated.

The actual dosage amount of the pharmaceutical compositions of the present invention administered to a recipient subject (e.g., a human or an animal) may be determined by physical and physiological factors such as body weight, severity of condition, the type of disease being treated, previous or concurrent therapeutic interventions, idiopathy of the patient, and by the route of administration. The practitioner responsible for administration will, in any event, determine the concentration of active ingredient(s) in the pharmaceutical compositions of the present invention and appropriate dose(s) for the individual subject.

The pharmaceutical compositions of the present invention may comprise various antioxidants to retard oxidation of one or more components. Additionally, the pharmaceutical compositions of the present invention further may comprise various antibacterial and antifungal agents, including, but not limited to, parabens (e.g., methylparabens, propylparabens), chlorobutanol, phenol, sorbic acid, thimerosal, or combinations thereof which may prevent the action of microorganisms.

One or more of the pharmaceutical compositions of the present invention, component of the pharmaceutical compositions of the present invention, and additional agents of the pharmaceutical compositions of the present invention may be formulated in buffered solution at a range of different pH values so that the composition may exist in neutral or salt form. Pharmaceutically acceptable salts include the acid-addition salts, for example, those formed with the free amino groups of a proteinaceous composition; those formed with inorganic acids such as, for example, hydrochloric or phosphoric acids; those formed with organic acids such as acetic, oxalic, tartaric, or mandelic. Salts formed with free carboxyl groups also may be derived from inorganic bases such as, for example, sodium, potassium, ammonium, calcium, or ferric hydroxides; or from organic bases such as isopropylamine, trimethylamine, histidine, or procaine.

The pharmaceutical compositions of the present invention should be stable under the conditions of manufacture, storage, and delivery, and preserved against the contaminating action of microorganisms, such as bacteria and fingi. It will be appreciated that endotoxin contamination should be kept minimally at a safe level, for example, less than 0.5 ng/mg protein.

In particular embodiments, prolonged absorption pharmaceutical compositions of the present invention that are suitable for injection may be brought about by the use of agents that delay absorption, such as, for example, aluminum monostearate, gelatin, or both.

Kits of the Present Invention.

Any of the compositions of the present invention described herein may be provided in a kit. A kit of the present invention may comprise a dendritic nitric oxide donor of the present invention and one or more additional, optional components such as, for example, a drug, another therapeutic agent, a diagnostic agent, a targeting agent, and an additional agent covalently coupled to and/or physically trapped in the dendritic nitric oxide donor. The kits of the present invention also may contain a means for delivering the formulation, such as, for example, a syringe for systemic administration, an inhaler or other pressurized aerosol canister, and the like.

The kits of the present invention may include a suitable aliquot of a dendritic nitric oxide donor of the present invention composed of a drug, another therapeutic agent, a diagnostic agent, a targeting agent and/or additional agent compositions of the present invention, chemically coupled to and/or physically trapped in the polymeric carrier, for example, as a guest molecule. The pharmaceutical compositions of the present invention present in the kits of the present invention may be packaged either in aqueous media or in lyophilized form. The container means of the kits of the present invention will generally include at least one vial, test tube, flask, bottle, syringe, or other container means, into which a component may be placed (e.g., as a suitable aliquot). When more than one component in the kits of the present invention is present, such kits also may contain a second, third or other additional container into which the additional components may be separately placed. However, various combinations of components may be placed in a single vial. The kits of the present invention also will typically include a means for containing the aerosol formulation, one or more components of an aerosol formulation, additional agents, and any other reagent containers in close confinement for commercial sale. Such containers may include injection or blow-molded plastic containers into which the desired vials are retained. The kits of the present invention may have a single container, or a distinct container for each compound.

When the components of the kits of the present invention are provided in one or more liquid solutions, the liquid solution is an aqueous solution, with a sterile aqueous solution being preferred. The components of the kits of the present invention, however, also may be provided as dried powder(s). When reagents and components are provided as a dry powder, the powder may be reconstituted by the addition of a suitable solvent. It is envisioned that the solvent also may be provided in another container means.

The container means will generally include at least one vial, test tube, flask, bottle, syringe, and/or other container means, into which a pharmaceutical composition of the present invention, a component of an aerosol formulation, and/or an additional agent formulation are suitably allocated. The kits of the present invention also may include a second container means for containing a sterile, pharmaceutically acceptable buffer and/or other diluent.

The kits of the present invention may include a means for containing the vials in close confinement for commercial sale, such as, for example, injection or blow-molded plastic containers into which the desired vials are retained.

Irrespective of the number or type of containers, the kits of the present invention also may include, or be packaged with, an instrument for assisting with the delivery of the aerosol formulation within the body of a human or an animal. Such an instrument may be a syringe, an inhaler, an air compressor, or any such medically approved delivery vehicle.

Medical Devices of the Present Invention.

In certain embodiments, the dendritic nitric oxide donors of the present invention may be incorporated on or within any medical device in which the release of NO may be beneficial, for example, blood-contacting devices. In certain embodiments, the dendritic nitric oxide donors of the present invention may be immobilized on the surface of a medical device or may be provided on the surface of a device through self-assembly. In certain embodiments, dendritic nitric oxide donors of the present invention comprising terminal amines may be attached to a surface-activated monolayer (SAM), and attached via amide bonds formed through acid chloride condensation. In certain embodiments, the dendritic nitric oxide donors of the present invention may be incorporated into a hydrogel matrix that can be polymerized on the surface of a medical device. Suitable hydrogels include those comprising poly(ethylene glycol), poly(lactic acid), poly(glycolic acid), or a combination thereof. In addition, the rate of degradation of these hydrogels may be tailored by formulating a copolymer (see generally Biomaterials Science: An Introduction to Materials in Medicine, B. D. Ratner et al. (Eds.) 66-69 (Academic Press 1996)). In certain embodiments, the dendritic nitric oxide donors of the present invention may be operatively attached to a medical device, including, but not limited to, a suture, a vascular implant, a stent, a heart valve, a drug pump, a drug-delivery catheter, an infusion catheter, a drug-delivery guidewire, or an implantable medical device.

To facilitate a better understanding of the present invention, the following examples of specific embodiments are given. In no way should the following examples be read to limit, or to define, the entire scope of the invention.

### EXAMPLES

Synthesis of Certain Dendritic Nitric Oxide Donor.

Specific examples of dendritic nitric oxide donors were synthesized using liquid phase peptide synthesis using methods found in “The Peptides,” supra, at 286-32. Each generation of dendrimer was formed using the method of J. S. Choi, et al., Bioconjugate Chemistry, 10(1):62-65 (1999). Polymer cores, in this example methoxypoly(ethylene glycol)-amine and diaminopoly(ethylene glycol), were used as the building blocks for synthesis of the dendritic nitric oxide donor. Each polymer core was reacted with four molar equivalents of Nα-Nε-di-FMOC-L-lysine in anhydrous N-N-dimethylformamide (DMF) in the presence of four molar equivalents each of N-hydroxybenzotriazole (HOBT), O-benzotriazol-1-YL-N,N,N′,N′-tetramethyluronium hexafluorophosphate (HBTU), and N,N-diisopropylethylamine (DIPEA). The resulting lysine copolymers were precipitated in ether, filtered, and then deprotected in 30% piperidine and precipitated and filtered a second time. This reaction scheme was repeated to form each subsequent generation of dendrimer.

A peptide synthesizer (Model 431A, Applied Biosystems, Foster City, Calif.) was used for the synthesis using solid phase peptide synthesis methods (see “The Peptides,” supra, at 284); a lysine resin (commercially available from Applied Biosystems); and four molar equivalents of Nα,Nε-di-FMOC-lysine (commercially available from Fluka). A ninhydrin assay was used to monitor the attachment of branching units to the core of the dendritic nitric oxide donor. In the case of a dendritic nitric oxide donor in which the branching unit monomer was lysine, such coupling exceeded 80%.

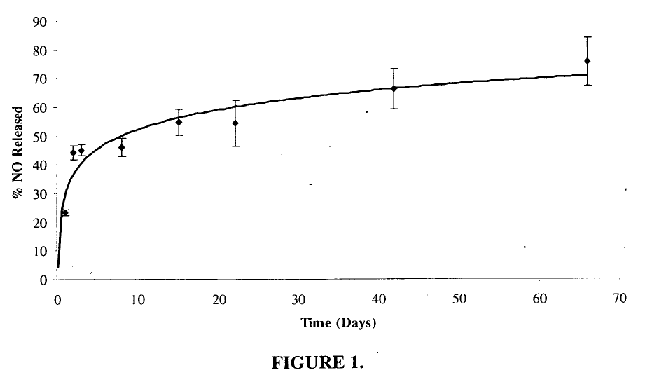

The resulting polymers were reacted with nitric oxide (NO) gas in water to form NO-nucleophile complexes or NO-donors, which are designed to release NO under physiological conditions. Coupling and deprotection reactions, as well as conversion of amines to diazeniumdiolate ions, were monitored by ninhydrin assay. NO release from each generation of dendritic nitric oxide donor species was determined by incubating dendrimers under physiological conditions and monitoring NO release by the Griess assay. In the case of a dendritic nitric oxide donor in which the branching unit monomer was lysine, upon reaction with NO, approximately 30%-70% of the primary amines present converted to diazeniumdiolate. Roughly 75%-90% of the coupled NO was released from each species of first-generation dendrimer within the first 48 hours (FIG. 1), each subsequent generation releasing over a more prolonged period of time with the addition of available NO-releasing moieties.

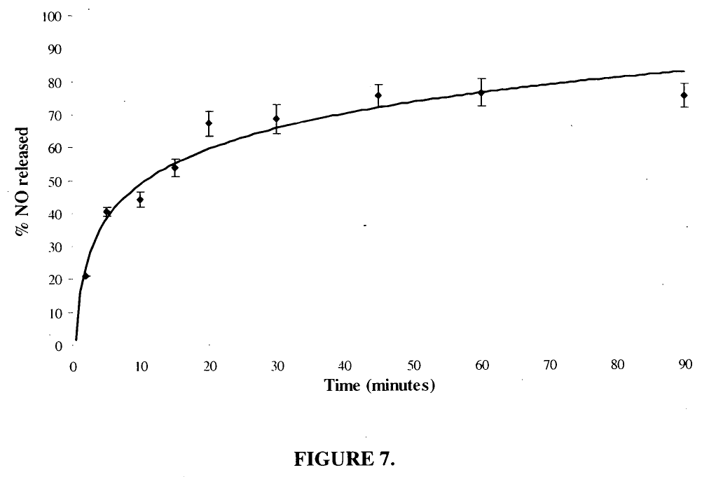

The Griess assay also was used to demonstrate the NO release profile of a diazeniumdiolate ion comprising three lysines (FIG. 5), a diazeniumdiolate ion comprising five lysines (FIG. 6), and a S-nitrosothiol comprising cysteine (FIG. 7).

Stimulation of Endothelial Cell Proliferation and Inhibition of Smooth Muscle Cell Proliferation by Certain Dendritic Nitric Oxide Donors.

To establish the success of a dendritic nitric oxide donor of the present invention in promoting endothelial cell growth Bovine aortic endothelial cells (BAECs; Clonetics, San Diego, Calif.) and Sprague-Dawley rat aortic smooth muscle cells (SDSMCs), passage 2-5, were used. The cells were maintained as follows. Dulbecco's modified Eagles's medium (DMEM; Sigma Chemical Co., St. Louis, Mo.) was prepared with 10% fetal bovine serum (FBS; BioWhitaker, Walkersville, Md.), 2 mM L-glutamine, 1 U/mL penicillin, and 100 mg/L streptomycin (GPS; Sigma Chemical Co., St. Louis, Mo.). Endothelial basal medium (EBM; Sigma Chemical Co., St. Louis, Mo.) was prepared with 10% endothelial medium supplement (Sigma Chemical Co., St. Louis, Mo.), which contained FBS, basic fibroblast growth factor (bFGF), heparin, epidermal growth factor, and hydrocortisone. BAECs were maintained on a mixture of EBM and DMEM (25/75 or 50/50 volume ratio) at 37° C. in a 5% CO₂ environment.

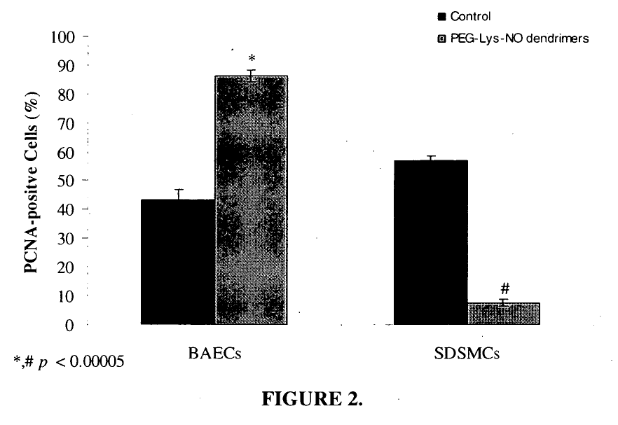

BAECs were seeded at a density of 10,000 cells/cm². A dendritic nitric oxide donor that released approximately 5.0 nmol NO per 1 mL cell culture media was added after 24 hours. An identical experiment was performed using SDSMCs. After 48 hours of culture in the presence of the the dendritic nitric oxide donor, cells were trypsinized and counted using a Coulter Counter. As shown in FIG. 2, the dendritic nitric oxide donor enhanced endothelial cell growth and inhibited smooth muscle cell proliferation. These experiments have been repeated with varying doses of NO, and proliferation was quantified using immunohistochemical staining for proliferating cell nuclear antigen, or PCNA, which stains cells in the S-phase of mitosis.

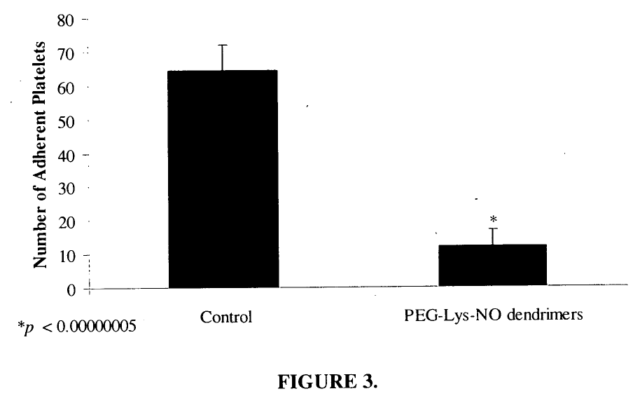

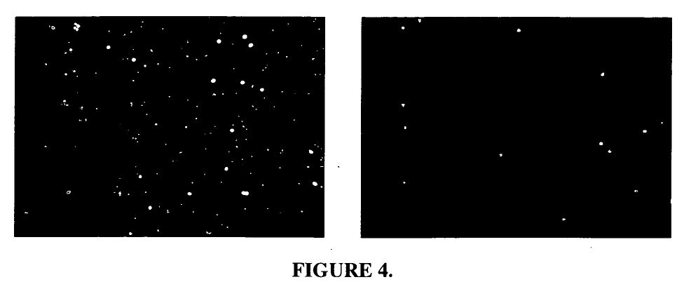

The affect of a dendritic nitric oxide donor on platelet aggregation was studied as follows. Blood was obtained from a healthy volunteer with 10 U/mL heparin (Sigma Chemical Co., St. Louis, Mo.). 10 μM mepacrine (Sigma Chemical Co., St. Louis, Mo.) was added for 20 minutes at 37° C. to fluorescently label the platelets. Glass slides were incubated with collagen I in 3% glacial acetic acid in distilled water (2.5 mg/mL) for 45 minutes in a humidified chamber at room temperature, and then rinsed gently with PBS. Labeled blood was incubated, for 30 minutes at 37° C., with either (i) the dendritic nitric oxide donor of the present invention that comprised an eight-arm PEG core, a 3rd generation branching unit, and NO, or (ii) a control that comprised the same compound without NO. The slides were then rinsed with PBS to remove all visible blood. The number of adherent platelets per field of view (200×) was determined using a fluorescent microscope (Zeiss Axiovert 135, Thomwood, N.Y.). As seen in FIGS. 3 and 4, the dendritic nitric oxide donor was able to inhibit platelet adhesion to collagen-coated slides (12.3±4.5 platelets per field of view) as compared to platelets exposed to the control (64.6±7.5 platelets per field of view, p<0.00000005).

Stimulation of Endothelial Cell Proliferation and Inhibition of Smooth Muscle Cell Proliferation by Targeting Certain Dendritic Nitric Oxide Donors.

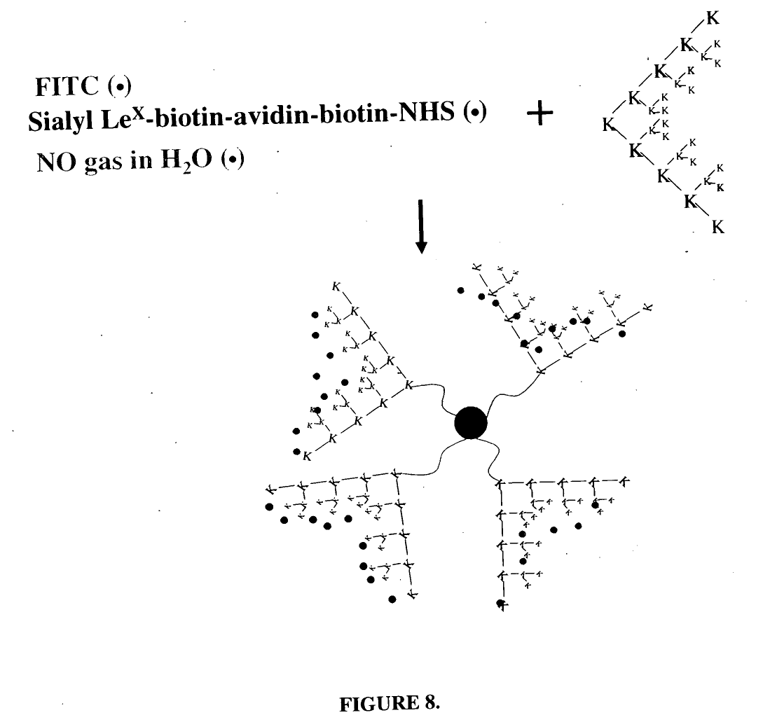

To demonstrate targeting, a dendritic nitric oxide donor conjugated to fluorescently labeled sialyl-Lewis-X was synthesized and studied as follows (FIG. 8). Lysine dendrons were reacted with fluorescein 5-isothiocyanate in dimethyl sulfoxide (DMSO) to fluorescently label the dendrons. Other dendrons were reacted with biotin-NHS for later conjugation of sialyl-Lewis-X-biotin using avidin as a linker, while others were reacted with NO gas in water. FITC conjugated dendrons, biotinylated dendrons, and NO-releasing dendrons were reacted with multi-armed PEG to form a species having a fluorescent tag and available biotin to bind the targeting molecule. Sialyl-Lewis-X was reacted with avidin in water, and then added to a solution of FITC-labeled biotinylated dendrimers to allow binding of sialyl-Lewis-X to the dendrimers.

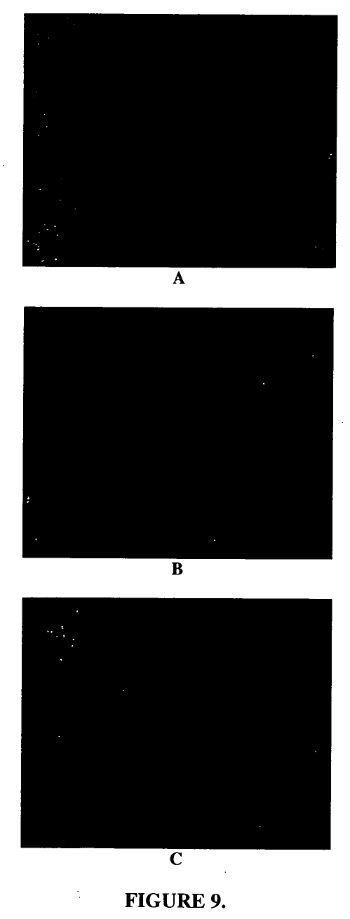

The dendritic nitric oxide donor having a fluorescently labeled sialyl-Lewis-X was then studied as follows. Human umbilical vein endothelial cells (HUVECs) were seeded in 6-well tissue culture plates at 20,000 cells/cm² and allowed to adhere for 24 hours. Cells were incubated with 5 μg/mL Interleukin-1β for 4 hours at 37° C., then exposed to either FITC-labeled sialyl-Lewis-X conjugated dendrimers (FIG. 9A) or FITC-labeled non-targeted dendrimers (FIG. 9C) for 30 minutes. As negative controls, a portion of the cells was not activated, and thus did not display elevated levels of E-selectin, and another set of cells were exposed to an E-selectin antibody after activation (FIG. 9B). The cells were then rinsed 3 times with PBS to remove non-adherent dendrimers and examined by fluorescence microscopy to determine the extent of binding. As shown in FIG. 9, the sialyl-Lewis-X conjugated dendritic nitric oxide donors preferentially bind HUVECs.

Therefore, the present invention is well adapted to attain the ends and advantages mentioned as well as those that are inherent therein. While numerous changes may be made by those skilled in the art, such changes are encompassed within the spirit of this invention as defined by the appended claims.

1. A dendritic nitric oxide donor having the formula:
[P]-[(A)_{y}]ₓ-[(NO)_{z}]_{q}
wherein P is a core that comprises a biocompatible polymer;
A is a branching unit monomer that comprises at least one end group capable of reversibly attaching NO;
(NO) is nitric oxide;
x, y, and z are positive integers greater than or equal to 1; and
q is a positive integer greater than or equal to y.

2. The dendritic nitric oxide donor of claim 1 wherein the dendritic nitric oxide donor comprises a metabolically produced form of the dendritic nitric oxide donor.

3. The dendritic nitric oxide donor of claim 1 wherein P comprises a functional group.

4. The dendritic nitric oxide donor of claim 1 wherein P comprises a functional group, wherein the functional group chosen from the group consisting of an amine group, a hydroxyl group, a N-hydroxysuccinimide ester, a carboxyl group, and combinations thereof.

5. The dendritic nitric oxide donor of claim 1 wherein P further comprises NO.

6. The dendritic nitric oxide donor of claim 1 wherein P comprises a metal.

7. The dendritic nitric oxide donor of claim 1 wherein P is chosen from the group consisting of polyethylene glycol, poly(ethylenamine), poly(amidoamine), polypropylenimine tetraamine, and a combination thereof.

8. The dendritic nitric oxide donor of claim 1 wherein P is chosen from the group consisting of methoxypoly(ethylene glycol)-amine, diaminopoly(ethylene glycol), PEG-N-hydroxysuccinimide ester monoacrylate, a multi-arm PEG, an mPEG-NHS, and a combination thereof.

9. The dendritic nitric oxide donor of claim 1 wherein A further comprises a functional group capable of releasing NO.

10. The dendritic nitric oxide donor of claim 1 wherein A further comprises a functional group capable of releasing NO chosen from the group consisting of an amine group, a carboxyl group, a thiol group, a hydroxyl group, and a combination thereof.

11. The dendritic nitric oxide donor of claim 1 wherein the end group of A is chosen from the group consisting of a primary amine, a thiol, a ferrous nitro complex, an organic nitrite, a nitrate, and a combination thereof.

12. The dendritic nitric oxide donor of claim 1 wherein the end group of A is capable of forming a NO-nucleophile complex.

13. The dendritic nitric oxide donor of claim 1 wherein the end group of A is capable of forming diazeniumdiolate ion.

14. The dendritic nitric oxide donor of claim 1 wherein the end group of A is capable of forming a NO-donating group.

15. The dendritic nitric oxide donor of claim 1 wherein the end group of A is capable of forming an S-nitrosothiol.

16. The dendritic nitric oxide donor of claim 1 wherein the end group of A is capable of forming a chemical species chosen from the group consisting of organic nitrites and nitrates, ferrous nitro complexes, sydnonimines, and combinations thereof.

17. The dendritic nitric oxide donor of claim 1 wherein A comprises an amino acid.

18. The dendritic nitric oxide donor of claim 1 further comprising a targeting agent.

19. The dendritic nitric oxide donor of claim 1 further comprising a targeting agent chosen from the group consisting of a protein, an antibody, an antibody fragment, a peptide, a cytokine, a growth factor hormone, a lymphokine, a nucleic acid that binds corresponding nucleic acids through base pair complementarity, a cellular receptor-targeting ligand, a fusogenic ligand, a nucleus-targeting ligand, an integrin receptor ligand, molecules that bind to a cell surface molecule, folic acid, and a combination thereof.

20. The dendritic nitric oxide donor of claim 1 further comprising a targeting agent that is capable of binding to a selectin.

21. The dendritic nitric oxide donor of claim 1 further comprising a targeting agent comprising sialyl-Lewis-X.

22. The dendritic nitric oxide donor of claim 1 further comprising a guest molecule.

23. The dendritic nitric oxide donor of claim 1 further comprising a guest molecule chosen from the group consisting of a drug, a therapeutic agent, a diagnostic agent, and a combination thereof.

24. The dendritic nitric oxide donor of claim 1 further comprising a guest molecule comprising 3-(5′-hydroxymethyl-2′-furyl)-1-benzyl indazole.

25. A kit comprising at least one dendritic nitric oxide donor, wherein the dendritic nitric oxide donor comprises a compound having the formula:
[P]-[(A)_{y}]ₓ-[(NO)_{z}]_{q}
wherein P is a core that comprises a biocompatible polymer;
A is a branching unit monomer that comprises at least one end group capable of reversibly attaching NO;
(NO) is nitric oxide;
x, y, and z are positive integers greater than or equal to 1; and
q is a positive integer greater than or equal to y.

26. The kit of claim 25 further comprising a drug, a therapeutic agent, a diagnostic agent, or a combination thereof.

27. The kit of claim 25 wherein the dendritic nitric oxide donor further comprises a targeting agent.

28. The kit of claim 25 wherein the dendritic nitric oxide donor further comprises a targeting agent chosen from the group consisting of a protein, an antibody, an antibody fragment, a peptide, a cytokine, a growth factor hormone, a lymphokine, a nucleic acid that binds corresponding nucleic acids through base pair complementarity, a cellular receptor-targeting ligand, a fusogenic ligand, a nucleus-targeting ligand, an integrin receptor ligand, molecules that bind to a cell surface molecule, folic acid, a selectin ligand, sialyl-Lewis-X, and a combination thereof.

29. The kit of claim 25 wherein the dendritic nitric oxide donor further comprises a guest molecule.

30. The kit of claim 25 wherein wherein P is chosen from the group consisting of polyethylene glycol, poly(ethylenamine), poly(amido amine), polypropylenimine tetraamine, methoxypoly(ethylene glycol)-amine, diaminopoly(ethylene glycol), PEG-N-hydroxysuccinimide ester monoacrylate, a multi-arm PEG, an mPEG-NHS, and a combination thereof.

31. The kit of claim 25 wherein A comprises an amino acid.

32. The kit of claim 25 further comprising a delivery means.

33. The kit of claim 25 further comprising a delivery means chosen from the group consisting of a syringe, an inhaler, pressurized aerosol canister, and a combination thereof.

34. The kit of claim 25 further comprising a container means.

35. The kit of claim 25 further comprising a container means chosen from a vial, a test tube, a flask, bottle, a syringe, and a combination thereof.

36. A medical device comprising at least one dendritic nitric oxide donor, wherein the dendritic nitric oxide donor comprises a compound having the formula:
[P]-[(A)_{y}]ₓ-[(NO)_{z}]_{q}
wherein P is a core that comprises a biocompatible polymer;
A is a branching unit monomer that comprises at least one end group capable of reversibly attaching NO;
(NO) is nitric oxide;
x, y, and z are positive integers greater than or equal to 1; and
q is a positive integer greater than or equal to y.

37. The medical device of claim 36 wherein the dendritic nitric oxide donor further comprises a targeting agent.

38. The medical device of claim 36 wherein the dendritic nitric oxide donor further comprises a targeting agent chosen from the group consisting of a protein, an antibody, an antibody fragment, a peptide, a cytokine, a growth factor hormone, a lymphokine, a nucleic acid that binds corresponding nucleic acids through base pair complementarity, a cellular receptor-targeting ligand, a fusogenic ligand, a nucleus-targeting ligand, an integrin receptor ligand, molecules that bind to a cell surface molecule, folic acid, a selectin ligand, sialyl-Lewis-X, and a combination thereof.

39. The medical device of claim 36 wherein the dendritic nitric oxide donor further comprises a guest molecule.

40. The medical device of claim 36 wherein the dendritic nitric oxide donor further comprises a guest molecule comprising 3-(5′-hydroxymethyl-2′-furyl)-1-benzyl indazole.

41. The medical device of claim 36 wherein the dendritic nitric oxide donor further comprises a guest molecule chosen from the group consisting of a drug, a therapeutic agent, a diagnostic agent, and a combination thereof.

42. The medical device of claim 36 wherein P is chosen from the group consisting of polyethylene glycol, poly(ethylenamine), poly(amidoamine), polypropylenimine tetraamine, methoxypoly(ethylene glycol)-amine, diaminopoly(ethylene glycol), PEG-N-hydroxysuccinimide ester monoacrylate, a multi-arm PEG, an mPEG-NHS, and a combination thereof.

43. The medical device of claim 36 wherein A comprises an amino acid.

44. The medical device of claim 36 wherein the dendritic nitric oxide donor is incorporated into a hydrogel.

45. The medical device of claim 36 wherein the dendritic nitric oxide donor is incorporated into a hydrogel comprising a polymer chosen from the group consisting of poly(ethylene glycol), poly(lactic acid), poly(glycolic acid), and combinations thereof.

46. The medical device of claim 36 wherein the medical device is chosen from the group consisting of a suture, a vascular implant, a stent, a heart valve, a drug pump, a drug-delivery catheter, an infusion catheter, a drug-delivery guidewire, an implantable medical device, and combinations thereof.

47. A method of delivering nitric oxide to a recipient subject comprising: providing a dendritic nitric oxide donor having the formula:
[P]-[(A)_{y}]ₓ-[(NO)_{z}]_{q}
wherein P is a core that comprises a biocompatible polymer;
A is a branching unit monomer that comprises at least one end group capable of reversibly attaching NO;
(NO) is nitric oxide;
x, y, and z are positive integers greater than or equal to 1;
q is a positive integer greater than or equal to y; and
administering the dendritic nitric oxide donor to a recipient subject, such that the dendritic nitric oxide donor releases NO in the recipient subject.

48. The method of claim 47 wherein the dendritic nitric oxide donor has a metabolically produced form.

49. The method of claim 47 wherein the recipient subject has a cardiovascular disease or condition.

50. The method of claim 47 wherein the recipient subject has a cardiovascular disease or condition chosen from the group consisting of restenosis, coronary artery disease, atherosclerosis, atherogenesis, cerebrovascular disease, angina, ischemic disease, congestive heart failure, pulmonary edema associated with acute myocardial infarction, thrombosis, high or elevated blood pressure in hypertension, platelet aggregation, platelet adhesion, smooth muscle cell proliferation, a vascular or nonvascular complication associated with the use of a medical device, a wound associated with the use of a medical device, vascular or nonvascular wall damage, peripheral vascular disease, neointimal hyperplasia following percutaneous transluminal coronary angiograph, and a combination thereof.

51. The method of claim 47 wherein the recipient subject has a pathological condition resulting from abnormal cell proliferation.

52. The method of claim 47 wherein the recipient subject has a disease selected from the group consisting of a cancer, a transplant rejection, an autoimmune disease, an inflammatory disease, a proliferative disease, a hyperproliferative disease, a vascular disease, a scar tissue, a wound contraction, and a combination thereof.

53. The method of claim 47 wherein the recipient subject has a pathological condition resulting from abnormal cell adherence.

54. The method of claim 47 wherein the dendritic nitric oxide donor further comprises a targeting agent.

55. The method of claim 47 wherein the dendritic nitric oxide donor further comprises a guest molecule.

56. The method of claim 47 wherein the dendritic nitric oxide donor further comprises a guest molecule chosen from the group consisting of a drug, a therapeutic agent, a diagnostic agent, and a combination thereof.

57. The method of claim 47 wherein the amount of dendritic nitric oxide donor is sufficient to provide a therapeutic amount of NO to the recipient subject.
